(19) United States
(12) Patent Application Publication
(10) Pub. No.: US 2024/0215899 A1
(43) Pub. Date: Jul. 4, 2024

(54) APPARATUS AND METHOD FOR CLASSIFYING HEART DISEASE USING MOBILENET

(71) Applicant: INDUSTRY-ACADEMIC COOPERATION FOUNDATION, CHOSUN UNIVERSITY, Gwangju (KR)

(72) Inventors: Youn-Tae KIM, Daejeon (KR); Jae-Hyo JUNG, Gwangju (KR); Si-Ho SHIN, Gwangju (KR); Min-Gu KANG, Gwangju (KR)

(73) Assignee: INDUSTRY-ACADEMIC COOPERATION FOUNDATION, CHOSUN UNIVERSITY, Gwangju (KR)

(21) Appl. No.: 18/558,110

(22) PCT Filed: Nov. 24, 2022
(86) PCT No.: PCT/KR2022/018722

§ 371 Date: Oct. 30, 2023

(30) Foreign Application Priority Data
Nov. 24, 2021 (KR) 10-2021-0163380
Nov. 22, 2022 (KR) 10-2022-0157248

(51) Int. Cl. A61B 5/347; A61B 5/00; G06N 3/0895

(52) U.S. Cl. CPC A61B 5/347; A61B 5/726; A61B 5/7264; A61B 5/7267; G06N 3/0895

(57) ABSTRACT

An apparatus for classifying heart disease using a MobileNet according to an embodiment of the present invention may comprise: an input unit for receiving a time-domain electrocardiogram signal; a wavelet transform unit for transforming the timedomain electrocardiogram signal into a frequency-domain electrocardiogram signal by using a wavelet transform; and a neural network for classifying the frequency-domain electrocardiogram signal as one of atrial fibrillation (AFIB), left bundle branch block beat (LBBB), normal sinus rhythm (NSR), or premature ventricular contraction (PVC), wherein the neural network may be a MobileNet trained using a training data set.

### TECHNICAL FIELD

The present disclosure relates to an apparatus and a method for classifying heart diseases using a MobileNet.

### BACKGROUND ART

An electrocardiogram (hereinafter referred to as an “ECG”) is used to measure rhythm of heartbeats represented by an electrical signal. The heart rate of a healthy person is maintained within a range of 60 to 100 beats per minute. When a person is exercising, tense, or excited, the heart of the person beats faster. In this case, there is usually no problem. However, when the heart beats irregularly for no reason, it is determined to be a problem with cardiac health. Such a symptom is referred to as heart disease. It is significantly important to detect heart disease in the early stage because manifestation of the heart disease may cause symptoms such as dizziness, fainting, chest pain, or difficulty in breathing and may result in heart attack.

Methods for diagnosing heart disease according to the related art cause patients, who should periodically visit a hospital and be periodically checked, to feel uncomfortable in terms of time and costs. An ECG, measured by smartwatches which have been widely used, is data measured within a short period of time of less than one minute and is unable to contribute to ascertain cardiovascular diseases including heart disease in advance. Methods for detecting heart disease include an analysis method for detecting premature ventricular contraction using RR intervals, an analysis method using discrete Fourier transformation, and an analysis method based on the Hilbert transformation.

Such heart disease diagnosis methods should be performed by experts and need to secure a significantly large amount of irregularly generated ECG data to achieve early detection of heart disease. However, when an ECG is measured for a long period of time, a large amount of data should be analyzed, and there are various factors taken into account for accurate diagnosis, such as movement of a person to be tested and a signal interference issue during ECG measurement in daily life. In addition, performance of hardware may be deteriorated due to a deep learning structure having a significantly large size.

As a related art, there is Korean Patent Publication No. 10-2008-0038512 (entitled “SYSTEM FOR PROVIDING DRIVER'S STRESS INDEX USING ECG,” published on May 7, 2008).

### SUMMARY OF INVENTION

### Technical Problem

An aspect of the present disclosure is to provide an apparatus and a method for classifying heart diseases using a MobileNet, which may prevent performance of hardware from being deteriorated due to a deep learning structure having a significantly large size, may periodically manage cardiovascular health in daily life using a smartphone or a smartwatch without a personal computer (PC) or a large-sized device, and may secure a sufficient amount of a training data set.

### Solution to Problem

According to an aspect of the present disclosure, an apparatus for classifying heart diseases using a MobileNet includes: an input unit configured to receive an electrocardiogram (ECG) signal in a time domain; a wavelet transformation unit configured to transform the ECG signal in the time domain into an ECG signal in a frequency domain; and a neural network configured to classify the ECG signal in the frequency domain as one of atrial fibrillation (AFIB), left bundle branch block beat (LBBB), normal sinus rhythm (NSR), and premature ventricular contraction (PVC). The neural network may be a MobileNet trained using a training dataset.

According to another aspect of the present disclosure, a method for classifying heart diseases using a MobileNet includes: a first operation in which an input unit receives an electrocardiogram (ECG) signal in a time domain; a second operation in which a wavelet transformation unit transforms the ECG signal in the time domain into an ECG signal in a frequency domain using wavelet transformation; and a third operation in which a neural network classifies the ECG signal in the frequency domain as one of atrial fibrillation (AFIB), left bundle branch block beat (LBBB), normal sinus rhythm (NSR), and premature ventricular contraction (PVC). The neural network may be a MobileNet trained using a training dataset.

According to another aspect of the present disclosure, a computer-readable recording medium, recording a program for executing the method on a computer, is provided.

### Advantageous Effects of Invention

According to an exemplary embodiment, an ECG signal in a time domain may be transformed into an ECG signal in a frequency domain using wavelet transformation, and the ECG signal in the frequency domain may be classified as one of atrial fibrillation (AFIB), left bundle branch block beat (LBBB), normal sinus rhythm (NSR), and premature ventricular contraction (PVC). Thus, performance of hardware may be prevented from being deteriorated due to a deep learning structure having a significantly large size, and cardiovascular health in daily life may be periodically managed by integration with a smartphone or a smartwatch, rather than a personal computer (PC) or a large-sized device.

In addition, according to an exemplary embodiment, by using a matching pursuit algorithm or by rotating each ECG signal in a time domain, the number of pieces of data may be increased to secure a sufficient amount of training dataset.

### BRIEF DESCRIPTION OF DRAWINGS

FIG. 1 is a block diagram of an apparatus for classifying heart diseases using a MobileNet according to an exemplary embodiment of the present disclosure.

FIG. 2 is a diagram illustrating data increased using a matching pursuit according to an exemplary embodiment of the present disclosure.

FIG. 3 is a diagram illustrating a wavelet-transformed ECG signal according to an exemplary embodiment of the present disclosure.

FIG. 4 is a graph illustrating classification accuracy of an apparatus for classifying heart diseases using a MobileNet according to an exemplary embodiment of the present disclosure, using 12-fold cross validation.

FIG. 5 is a flowchart illustrating a method for classifying heart diseases using a MobileNet according to an exemplary embodiment of the present disclosure.

FIG. 6 is a block diagram of a computer device which may fully or partially implement an apparatus for classifying heart diseases using a MobileNet according to an exemplary embodiment of the present disclosure.

### BEST MODE FOR INVENTION

Hereinafter, example embodiments of the present disclosure will be described in detail with reference to the accompanying drawings. The present disclosure may, however, be embodied in many different forms, and should not be construed as being limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

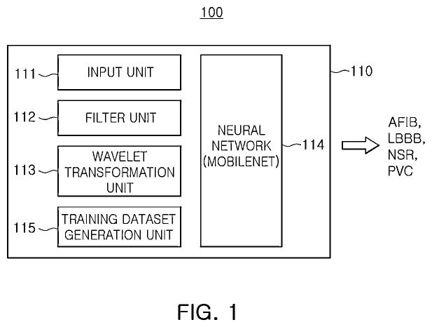

FIG. 1 is a block diagram of an apparatus for classifying heart diseases using a MobileNet according to an exemplary embodiment.

As illustrated in FIG. 1, an apparatus 100 for classifying heart diseases using a MobileNet according to an exemplary embodiment may include an input unit 111, a filter unit 112, a wavelet transformation unit 113 and a neural network 114.

For example, the input unit 111 may receive an ECG signal in a time domain and may transmit the received ECG signal to the filter unit 112.

The filter unit 112 may perform low-pass filtering on the ECG signal in the time domain received from the input unit 111. A cutoff frequency of the low-pass filtering may be 130 Hz. A noise correction effect of 91.79% may be obtained through the filter unit 112 having such a cutoff frequency.

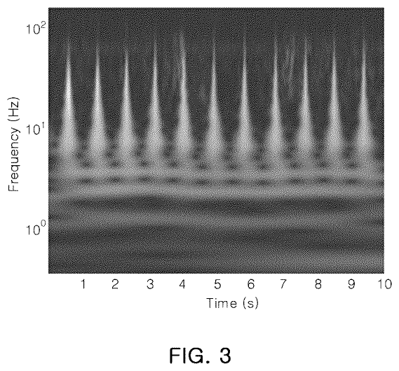

The wavelet transformation unit 113 may transform the ECG signal in the time domain into an ECG signal in a frequency domain using wavelet transformation. The transformed ECG signal in the frequency domain may be transmitted to the neural network 114. An example of the wavelet-transformed ECG signal according to an exemplary embodiment is illustrated in FIG. 3.

Then, the neural network 114 may classify the ECG signal in frequency domain as one of atrial fibrillation (AFIB), left bundle branch block beat (LBBB), normal sinus rhythm (NSR), and premature ventricular contraction (PVC).

According to an exemplary embodiment, the neural network 114 may be a MobileNet trained using a training dataset.

The above-mentioned MobileNet is a lightened deep learning model, and is a convolution neural network (CNN) structure designed for use in a place in which computer performance is limited or battery performance is important. Such a MobileNet may include known layers such as an input layer, a 2D layer, a normalization layer, a max pooling layer, a fully connected layer, and a soft max layer to classify the ECG signal in the frequency domain as one of atrial fibrillation (AFIB), left bundle branch block beat (LBBB), normal sinus rhythm (NSR), and premature ventricular contraction (PVC). Since an internal structure and functions of such a MobileNet are well known in the art, detailed descriptions thereof will be omitted.

According to an exemplary embodiment, the apparatus 100 may further include a training dataset generation unit 115 generating a training dataset for training of the above-described neural network 114.

The training dataset generation unit 115 may generate a second number of ECG signals in the time domain from a first number of ECG signals in the time domain to increase the number of pieces of data, and may then transform the second number of ECG signals in the time domain into an ECG signal in the frequency domain using wavelet transformation. The second number may be a value, greater than the first number.

When the neural network 114 is trained, accuracy may be improved as the number of pieces of data increases. Therefore, according to an exemplary embodiment, by using a matching pursuit algorithm or rotating each of the ECG signals in the time domain to increase the number of ECG signals, the number of pieces of data may be increased. Alternatively, both of the above-described two methods may be used to increase the number of pieces of data.

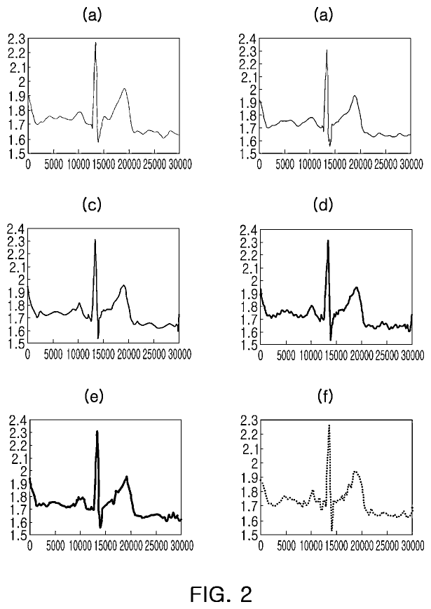

The above-described matching pursuit algorithm may be an algorithm which may similarly copy ECG data. The ECG data may be increased ten times using such a matching pursuit algorithm. (a) to (f) of FIG. 2 illustrate data increased using a matching pursuit according to an exemplary embodiment.

In addition, each of the ECG signals in the time domain may be rotated to increase the number of pieces of data. The rotation of the ECG signals in the time domain may cause an ECG signal in the time domain before the rotation and an ECG signal in the time domain after the rotation to be recognized as different types of data by the neural network 114.

The neural network 114 may be trained based on the training dataset generated by the above-described training dataset generation unit 115.

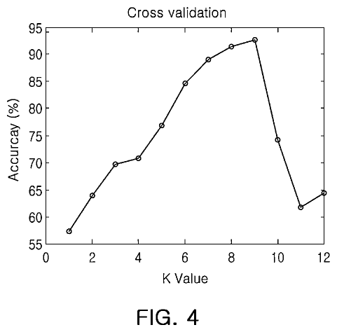

FIG. 4 is a diagram illustrating classification accuracy of an apparatus for classifying heart diseases using a MobileNet according to an exemplary embodiment, using 12-fold cross validation.

In K-fold cross validation, data may be divided into K groups, one of the K groups may be extracted and used as a test set, and the remaining (K-1) groups may be used as training sets. When a test is repeated K number of times, each test may obtain a single classification accuracy and may then obtain an average K result to obtain final performance of classification. In the present disclosure, the classification accuracy was measured using 12-fold cross validate, and a result of the measurement was 92.65% in 9-fold.

As described above, according to an exemplary embodiment, an ECG signal in a time domain may be transformed into an ECG signal in a frequency domain using wavelet transformation, and the ECG signal in the frequency domain may be classified as one of atrial fibrillation (AFIB), left bundle branch block beat (LBBB), normal sinus rhythm (NSR), and premature ventricular contraction (PVC). Thus, performance of hardware may be prevented from being deteriorated due to a deep learning structure having a significantly large size, and cardiovascular health in daily life may be periodically managed by integration with a smartphone or a smartwatch, rather than a personal computer (PC) or a large-sized device.

In addition, according to an exemplary embodiment, by using a matching pursuit algorithm or by rotating each ECG signal in a time domain, the number of pieces of data may be increased to secure a sufficient amount of training dataset.

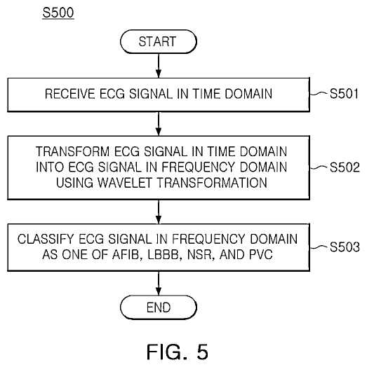

FIG. 5 is a flowchart illustrating a method for classifying heart diseases using a MobileNet according to an exemplary embodiment.

Hereinafter, a method for classifying heart diseases using a MobileNet according to an exemplary embodiment will be described in detail with reference to FIGS. 1 to 5. However, duplicate descriptions as those provided above with reference to FIGS. 1 to 4 will be omitted for clarity of the present disclosure.

The method for classifying heart diseases using a MobileNet according to an exemplary embodiment may start with operation S501 in which the input unit 111 receives an ECG signal in a time domain.

According to an exemplary embodiment, as described above, the ECG signal in the time domain may be subjected to low-pass filtering, and a cutoff frequency of the low-pass filtering may be 130 Hz.

In operation S502, the wavelet transformation unit 113 may transform the ECG signal in the time domain into an ECG signal in a frequency domain using wavelet transformation. The transformed ECG signal in the frequency domain may be transmitted to the neural network 114.

In operation S503, the neural network 114 may classify the ECG signal in the frequency domain as one of atrial fibrillation (AFIB), left bundle branch block beat (LBBB), normal sinus rhythm (NSR), and premature ventricular contraction (PVC).

According to an exemplary embodiment, the neural network 114 may be a MobileNet trained using a training dataset.

As described above, the above-mentioned MobileNet is a lightened deep learning model, and is a convolution neural network (CNN) structure designed for use in a place in which computer performance is limited or battery performance is important. Such a MobileNet may include known layers such as an input layer, a 2D layer, a normalization layer, a max pooling layer, a fully connected layer, and a soft max layer to classify the ECG signal in the frequency domain as one of atrial fibrillation (AFIB), left bundle branch block beat (LBBB), normal sinus rhythm (NSR), and premature ventricular contraction (PVC).

According to an exemplary embodiment, the training dataset generation unit 115 generating a training dataset for training of the above-described neural network 114 may be further provided.

The training dataset generation unit 115 may generate a second number of ECG signals in the time domain from a first number of ECG signals in the time domain to increase the number of pieces of data, and may then transform the second number of ECG signals in the time domain into an ECG signal in the frequency domain using wavelet transformation. The second number may be a value, greater than the first number.

When the neural network 114 is trained, accuracy may be improved as the number of pieces of data increases. Therefore, according to an exemplary embodiment, by using a matching pursuit algorithm or rotating each of the ECG signals in the time domain to increase the number of ECG signals, the number of pieces of data may be increased. Alternatively, both of the two above-described methods may be used to increase the number of pieces of data.

The above-described matching pursuit algorithm may be an algorithm which may similarly copy ECG data. The ECG data may be increased ten times using such a matching pursuit algorithm. (a) to (f) of FIG. 2 illustrate data increased using a matching pursuit according to an exemplary embodiment.

In addition, each of the ECG signals in the time domain may be rotated to increase the number of pieces of data. The rotation of the ECG signals in the time domain may cause an ECG signal in the time domain before the rotation and an ECG signal in the time domain after the rotation to be recognized as different types of data by the neural network 114.

The neural network 114 may be trained based on the training dataset generated by the above-described training dataset generation unit 115.

As described above, according to an exemplary embodiment, an ECG signal in a time domain may be transformed into an ECG signal in a frequency domain using wavelet transformation, and the ECG signal in the frequency domain may be classified as one of atrial fibrillation (AFIB), left bundle branch block beat (LBBB), normal sinus rhythm (NSR), and premature ventricular contraction (PVC). Thus, performance of hardware may be prevented from being deteriorated due to a deep learning structure having a significantly large size, and cardiovascular health in daily life may be periodically managed by integration with a smartphone or a smartwatch, rather than a personal computer (PC) or a large-sized device.

In addition, according to an exemplary embodiment, by using a matching pursuit algorithm or by rotating each ECG signal in a time domain, the number of pieces of data may be increased to secure a sufficient amount of training dataset.

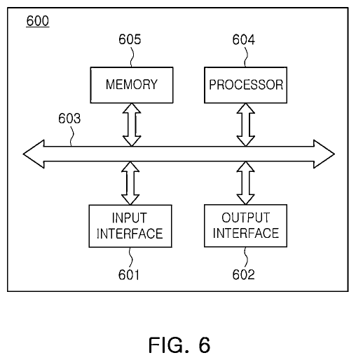

FIG. 6 is a block diagram of a computer device which may fully or partially implement an apparatus for classifying heart diseases using a MobileNet according to an exemplary embodiment, and the computer device may be applied to the apparatus 100 for classifying heart diseases using a MobileNet illustrated in FIG. 1.

As illustrated in FIG. 6, the computer device 600 may include an input interface 601, an output interface 602, a processor 604, and a memory 605, and the input interface 601, the output interface 602, the processor 604, and the memory 605 may be connected to each other through a system bus 603.

In an exemplary embodiment, the memory 605 may be used to store a program, an instruction, or a code, and the processor 604 may execute the program, the instruction, or the code stored in the memory 605. Also, the processor 604 may control the input interface 601 to receive a signal and may control the output interface 602 to transmit a signal. The memory 605 may include a read-only memory and a random access memory, and may provide an instruction and data to the processor 604.

In an exemplary embodiment, it should be understood that the processor 604 may be a central processing unit (CPU), and may also be other general-purpose processor or a digital signal processor (DSP), an application specific integrated circuit (ASIC), a field programmable gate array (FPGA) or other programmable logic device, discrete gate or transistor logic device, discrete hardware components, or the like. The general-purpose processor may be a microprocessor, or the processor may be any processor according to the related art, or the like.

In an implementation process, the method performed in each device of FIG. 1 may be implemented by an integrated logic circuit of hardware in the processor 604 or an instruction in the form of software. The content of the method disclosed in relation to exemplary embodiments may be implemented to be performed and completed by a hardware processor, or may be implemented to be performed and completed by a combination of hardware and software modules of a processor. The software module may be stored in a memory such as a random access memory, a flash memory, a read-only memory, a programmable read-only memory or an electrically erasable programmable memory, a register, or the like, and the processor 604 may read information in the memory 605 and may be coupled to hardware implement the content of the above-described method.

While example embodiments have been shown and described above, it will be apparent to those skilled in the art that modifications and variations could be made without departing from the scope of the present disclosure as defined by the appended claims.

1. An apparatus for classifying heart diseases using a MobileNet, the apparatus comprising:
an input unit configured to receive an electrocardiogram (ECG) signal in a time domain;
a wavelet transformation unit configured to transform the ECG signal in the time domain into an ECG signal in a frequency domain; and
a neural network configured to classify the ECG signal in the frequency domain as one of atrial fibrillation (AFIB), left bundle branch block beat (LBBB), normal sinus rhythm (NSR), and premature ventricular contraction (PVC),
wherein the neural network is a MobileNet trained using a training dataset.

2. The apparatus of claim 1, further comprising:
a filter unit configured to perform low-pass filtering on the ECG signal in the time domain,
wherein a cutoff frequency of the low-pass filtering is 130 Hz.

3. The apparatus of claim 1, further comprising:
a training dataset generation unit configured to generate the training dataset,
wherein the MobileNet performs training using the training dataset.

4. The apparatus of claim 3, wherein
the training dataset generation unit increases the number of pieces of data by generating a second number of ECG signals in the time domain from a first number of ECG signals in the time domain, the second number being greater than the first number, and
the second number of ECG signals in the time domain are transformed into ECG signals in the frequency domain using wavelet transformation.

5. The apparatus of claim 4, wherein
the training dataset generation unit increases the number of pieces of data by using a matching pursuit algorithm or by rotating each of the first number of ECG signals in the time domain.

6. A method for classifying heart diseases using a MobileNet, the method comprising:
a first operation in which an input unit receives an electrocardiogram (ECG) signal in a time domain;
a second operation in which a wavelet transformation unit transforms the ECG signal in the time domain into an ECG signal in a frequency domain using wavelet transformation; and
a third operation in which a neural network classifies the ECG signal in the frequency domain as one of atrial fibrillation (AFIB), left bundle branch block beat (LBBB), normal sinus rhythm (NSR), and premature ventricular contraction (PVC),
wherein the neural network is a MobileNet trained using a training dataset.

7. The method of claim 6, further comprising:
an operation in which a filter unit performs a low-pass filtering on the ECG signal in the time domain,
wherein a cutoff frequency of the low-pass filtering is 130 Hz.

8. The method of claim 6, comprising:
an operation in which a training dataset generation unit generates the training dataset; and
an operation in which the MobileNet performs training using the training dataset.

9. The method of claim 8, wherein
the operation in which the training dataset generation unit generates the training dataset comprises:
increasing the number of pieces of data by generating a second number of ECG signals in the time domain from a first number of ECG signals in the time domain, the second number being greater than the first number; and
transforming the second number of ECG signals in the time domain into ECG signals in the frequency domain using wavelet transformation.

10. The method of claim 9, wherein
the increasing the number of pieces of data includes increasing the number of pieces of data by using a matching pursuit algorithm or by rotating each of the first number of ECG signals in the time domain.
